SEQUENCE LISTING (6 sequences)

SEQ ID NO: 1 - DNA, 234 bp, Homo sapiens
gcagatagtggtgaaggtgactttctagctgaaggaggaggcgtgcgtggcccaagggttgtggaaagacatcaatctgcctgcaaagattcagactggcccttctgctctgatgaagactggaactacaaatgcccttctggctgcaggatgaaagggttgattgatgaagtcaatcaagattttacaaacagaataaataagctcaaaaattcactatttgaatatcagaag

SEQ ID NO: 2 - DNA, 240 bp, Homo sapiens
gctcgtccagccaaagcagctgccactcaaaagaaactagaaagaaaagcccctgatgctggaggctgtcttcacgctgacccagacctgggggtgttgtgtcctacaggatgtcagttgcaagaggctttgctacaacaggaaaggccaatcagaaatagtgttgatgagttaaataacaatgtggaagctgtttcccagacctcctcttcttcctttcagtacatgtatttgctgaaa

SEQ ID NO: 3 - DNA, 186 bp, Homo sapiens
tatgttgctaccagagacaactgctgcatcttagatgaaagattcggtagttattgtccaactacctgtggcattgcagattccctgtctacttatcaaaccaaagtagacaaggatctacagtctttggaagacatcttacatcaagttgaaaacaaaacatcagaagtcaaacagctgataaaa

SEQ ID NO: 4 - protein, 78 aa, Homo sapiens
ADSGEGDFLAEGGGVRGPRVVERHQSACKDSDWPFCSDEDWNYKCPSGCRMKGLIDEVNQDFTNRINKLKNSLFEYQK

SEQ ID NO: 5 - protein, 80 aa, Homo sapiens
ARPAKAAATQKKVERKAPDAGGCLHADPDLGVLCPTGCQLQEALLQQERPIRNSVDELNNNVEAVSQTSSSSFQYMYLLK

SEQ ID NO: 6 - protein, 62 aa, Homo sapiens
YVATRDNCCILDERFGSYCPTTCGIADFLSTYQTKVDKDLQSLEDILHQVENKTSEVKQLIK

(19) United States
(12) Patent Application Publication
(10) Pub. No.: US 2006/0019897 A1
(43) Pub. Date: Jan. 26, 2006

(54) Anti-angiogenic polypeptides

(76) Inventors: Carolyn Staton, Sheffield (GB); Claire Lewis, Sheffield (GB)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/188,576

(22) Filed: Jul. 25, 2005

Related U.S. Application Data
(62) Division of application No. 10/276,014, filed on Aug. 26, 2003, now abandoned.

(30) Foreign Application Priority Data
May 13, 2000 (GB) 0011464.5
Jun. 14, 2000 (GB) 0014370.1
Nov. 9, 2000 (GB) 0027396.1

(51) Int. Cl. C07K 14/75; C07H 21/04; C12P 21/04; A61K 38/36

(52) U.S. Cl. 514/12; 435/69.6; 435/320.1; 435/325; 536/23.5; 530/382

(57) ABSTRACT

The invention relates to anti-angiogenic effects of polypeptides derived from fibrinogen.

The invention relates to polypeptides with anti-angiogenic effects.

Angiogenesis, the development of new blood vessels from an existing vascular bed, is a complex multistep process that involves the degradation of components of the extracellular matrix and then the migration, proliferation and differentiation of endothelial cells to form tubules and eventually -new vessels. Angiogenesis is important in normal physiological processes including, by example and not by way of limitation, embryo implantation; embryogenesis and development; and wound healing.

Angiogenesis is also involved in pathological conditions such as tumour cell growth and non-cancerous conditions such as ophthalmological conditions for example, neovascular glaucoma; diabetic retinopathy; age-related macular degeneration, pterygium; retinopathy of prematurity; choroidal and other intraocular disorders

Angiogenesis is also involved in pathological conditions such as atherosclerosis; haemangioma; haemangioendothelioma; warts; hair growth; Kaposi's sarcoma; scar keloids; allergic oedema; dysfunctional uterine bleeding; follicular cysts; ovarian hyperstimulation; endometriosis; peritoneal sclerosis, adhesion formation; obesity; osteornyelitis; pannus growth; osteophyte formation; inflammatory and infectious processes (eg hepatitis, pneumonia, glomerulonephritis), asthma, nasal polyps, transplantation, liver regeneration; thyroiditis, thyroid enlargement; and lymphoproliferative disorders.

The vascular endothelium is normally quiescent. However upon activation endothelial cells proliferate and migrate to form microtubules which will ultimately form a capillary bed to supply blood to developing tissues and, of course, a growing tumour. A number of growth factors have been identified which promote/activate endothelial cells to undergo angiogenesis. These include, by example and not by way of limitation; vascular endothelial growth factor (VEGF); transforming growth factor (TGFb); acidic and basic fibroblast growth factor (aFGF and bFGF); and platelet derived growth factor (PDGF) (1,2).

VEGF is a an endothelial cell-specific growth factor which has a very specific site of action, namely the promotion of endothelial cell proliferation, migration and differentiation. VEGF is a dimeric complex comprising two identical 23 kD polypeptides. The monomeric form of VEGF can exist as four distinct polypeptides of different molecular weight, each being derived from an alternatively spliced mRNA. Of the four monomeric forms, two exist as membrane bound VEGF and two are soluble. VEGF is expressed by a wide variety of cell/tissue types including embryonal tissues; proliferating keratinocytes; macrophages; tumour cells. Studies (2) have shown VEGF is highly expressed in many tumour cell-lines including glioma and AIDS associated Karposi's sarcoma. VEGF activity is mediated through VEGF specific receptors expressed by endothelial cells and tumour cells. Indeed the VEGF receptor is up-regulated in endothelial cells which infiltrate tumours thereby promoting tumour cell growth.

bFGF is a growth factor which functions to stimulate the proliferation of fibroblasts and endothelial cells. bFGF is a single polypeptide chain with a molecular weight of 16.5 Kd. Several molecular forms of bFGF have been discovered which differ in the length at their amino terminal region. However the biological function of the various molecular forms appears to be the same. bFGF is produced by the pituitary gland and is encoded by a single gene located on human chromosome 4.

A number of endogenous inhibitors of angiogenesis have been discovered, examples of which are angiostatin and endostatin, which are formed by the proteolytic cleavage of plasminogen and collagen XVIII respectively. Both of these factors have been shown to suppress the activity of pro-angiogenic growth factors such as vascular VEGF and bFGF. Both of these factors suppress endothelial cell responses to VEGF and bFGF in vitro, and reduce the vascularisation and growth of experimental tumours in animal models.

We have discovered a potent, new inhibitor of angiogenesis which is a proteolytic fragment of fibrinogen.

Fibrinogen, the soluble circulating precursor of fibrin, is a dimeric molecule containing pairs of non-identical chains, (ie the α-, β- and γ-chains). These are arranged as three discrete domains, the two outer D-domains and the central E-domain (4). Fibrinogen can be digested either by plasmin or thrombin.

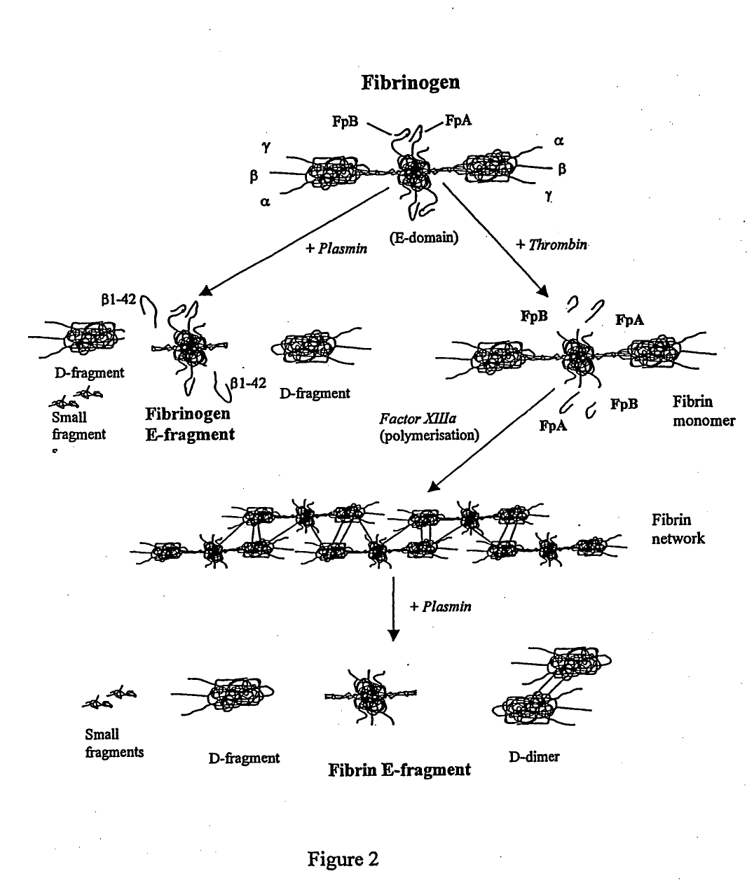

The first step in plasmin cleavage of fibrinogen is the cleavage of the a chain C-terminal domain. Plasmin then cleaves the two D domains from the one E domain (consisting of the NH2 terminal regions of the α-, β- and γ-chains held together by disulphide bonds) and numerous smaller fragments including a small peptide, beta 1-42 (amino terminal of the β-chain (5). Thrombin, on the other hand, produces a fibrin monomer and two copies of fibrinopeptides A and B (see FIG. 2) (4). Fibrinogen has been shown to accumulate around leaky blood vessels in solid tumours (5), Fibrinogen has also been shown to polymerise at host-tumour interface to form fibrin networks that promote tumour angiogenesis by supporting the adhesion, migration, proliferation and differentiation of endothelial cells (7).

The fibrin E-fragment (FnE-fragment), produced by the proteolytic cleavage of fibrin, stimulates angiogenesis in the chorioallantoic membrane assay (8). Furthermore, the amount of this protein present in invasive breast carcinomas positively corrolates with the degree of tumour vascularity (5).

WO99/45135 describes polypeptides having, amongst other things, angiogenic activity. The polypeptides are referred to as Fibrinogen Domain Related (FDRG) because of a conserved carboxyl terminal region found in a number of polypeptides (eg fibrinogen, angiopoietin, ficolin). This family of proteins have both a conserved structure and function. The polypeptide members of the FDRG family are distinguished from one another by unique variable amino-terminal regions. FDRG family members are implicated in a number of cellular processes including; modulation of angiogenesis, modulation of haematopoiesis, modulation of the proliferation, development or differentiation of adipocytes, modulation of insulin sensitivity and/or insulin responsiveness. However little, if any confirmatory experimental evidence is presented to fully corroborate these functions of the FDRG family of proteins. In addition the FDRG fragments disclosed in WO99/45135 are located in the carboxy-terminal region of the γ chain and therefore not part of the E fragment.

### STATEMENTS OF INVENTION

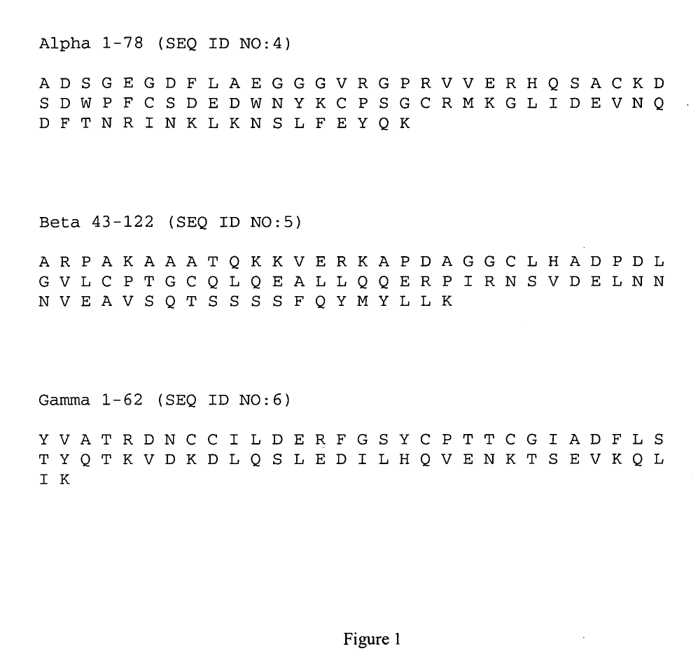

According to a first aspect of the invention there is provided a nucleic acid molecule comprising DNA sequences selected from:
i) a fragment of the DNA sequence encoding amino acids 1-78 of the α-chain of fibrinogen; amino acids 43-122 of the β-chain of fibrinogen; and amino acids 1-62 of the γ-chain of fibrinogen as represented in FIG. 1
ii) DNA sequences which hybridise to the sequences presented in (i) which encode fibrinogen E which has anti-angiogenic activity; and
iii) DNA sequences which are degenerate as a result of the genetic code to the DNA sequences defined in (i) and (ii).

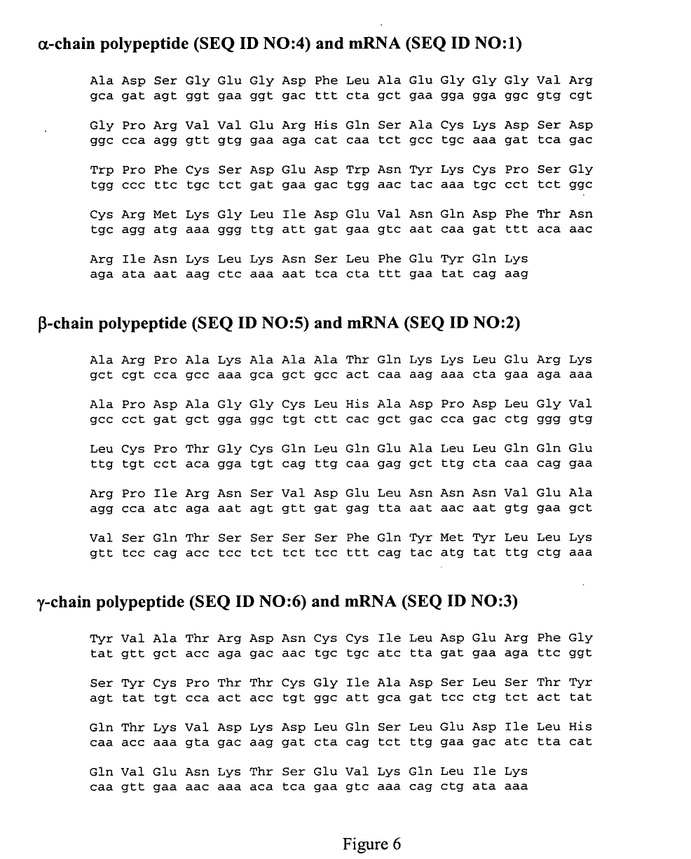

According to a further aspect of the invention there is provided a nucleic acid molecule comprising DNA sequences selected from:
i) the DNA sequences as represented in FIG. 6
ii) DNA sequences which hybridise to the sequences presented in FIG. 6 which encode a polypeptide having anti-angiogenic activity; and
iii) DNA sequences which are degenerate as a result of the genetic code to the DNA sequences defined in (i) and (ii).

In a preferred embodiment of the invention there is provided an isolated nucleic acid molecule which anneals under stringent hybridisation conditions to the sequences described in (i), (ii) and (iii) above.

Stringent hybridisation/washing conditions are well known in the art. For example, nucleic acid hybrids that are stable after washing in 0.1×SSC, 0.1% SDS at 60° C. It is well known in the art that optimal hybridisation conditions can be calculated if the sequence of the nucleic acid is known.

The DNA sequence of fibrinogen is known and can be found in the NCBI website at http://ncbi.nlm.nih.gov using appropriate search terms.

According to a second aspect of the invention there is provided a polypeptide encoded by the nucleic acid according to the invention.

In a preferred embodiment of the invention said polypeptide is fibrinogen E.

In a further preferred embodiment of the invention fibrinogen E comprises the NH2 domains of the α, β and γ polypeptides.

In a still further preferred embodiment of the invention said fibrinogen E comprises amino acids 1 to 78 of the α-chain and amino acids 43 to 122 of the β-chain; and amino acids 1 to 62 of the γ-chain, as represented in FIG. 1.

In yet still a further preferred embodiment of the invention polypeptides comprising fibrinogen E is/are modified by deletion, addition or substitution of at least one amino acid residue. Ideally said modification enhances the antagonistic effects of fibrinogen E with respect to the inhibition of angiogenesis.

It will be apparent to one skilled in the art that modification to the amino acid sequence of polypeptides comprising fibrinogen E could enhance the binding and/or stability of the fibrinogen E with respect to its target sequence (e.g. VEGF and/or bFGF). In addition, modification of fibrinogen E may also increase the in vivo stability of the fragment thereby reducing the effective amount of fragment necessary to inhibit angiogenesis. This would advantageously reduce undesirable side effects which may result in vivo.

Alternatively, or preferably, said modification includes the use of modified amino acids in the production of recombinant or synthetic forms of fibrinogen E.

It will be apparent to one skilled in the art that modified amino acids include, by way of example and not by way of limitation, 4-hydroxyproline, 5-hydroxylysine, N⁶-acetyllysine, N⁶-methyllysine, N⁶,N⁶-dimethyllysine, N⁶,N⁶,N⁶-trimethyllysine, cyclohexyalanine, D-amino acids, ornithine. The incorporation of modified amino acids will confer advantageous properties on fibrinogen E fragments. For example, the incorporation of modified amino acids may increase the affinity of the fragment for its binding site, or the modified amino acids may confer increased in vivo stability on the fragment thus allowing a decrease in the effective amount of therapeutic fragment administered to a patient.

According to a further aspect the invention there is provided a therapeutic composition comprising fibrinogen E. In a preferred embodiment of the invention said therapeutic composition modulates angiogenesis. Preferably said modulation is the inhibition of angiogenesis. Preferably said inhibition relates to endothelial cell stimulated angiogenesis.

A number of conditions would benefit from an inhibition of angiogenesis. For example, ophthalmological conditions such as, neovascular glaucoma; diabetic retinopathy; age-related macular degeneration, pterygium; retinopathy of prematurity; choroidal and other intraocular disorders. Also, atherosclerosis; haemangioma; haemangioendothelioma; warts; Kaposi's sarcoma; scar keloids; allergic oedema; dysfunctional uterine bleeding; follicular cysts; ovarian hyperstimulation,; endometriosis; peritoneal sclerosis, adhesion formation; obesity; osteomyelitis; pannus growth; osteophyte formation; inflammatory and infectious processes (eg hepatitis, pneumonia, glomerulonephritis), asthma, nasal polyps, transplantation, liver regeneration; thyroiditis, thyroid enlargement; and lymphoproliferative disorders.

Alternatively, or preferably, said inhibition is the inhibition of macrophage and/or tumour cell stimulated angiogenesis.

In a further preferred embodiment of the invention said inhibition is mediated by the inhibition of pro-angiogenic factors. Ideally these are either intracellular or cell surface receptors.

More preferably still, said inhibition is mediated via inhibition of the activity of pro-angiogenic growth factors. Ideally said growth factors are selected from: VEGF, bFGF; aFGF; TGFβ; PDGF.

According to a further aspect of the invention there is provided the use of fibrinogen E in the manufacture of a medicament for use in the treatment of cancer.

Polypeptides which comprise fibrinogen E can be manufactured by in vitro peptide synthesis using standard peptide synthesis techniques. Alternatively, or preferably, fibrinogen and/or polypeptides which comprise fibrinogen E can be manufactured by recombinant techniques which are well known in the art.

According to a further aspect of the invention there is provided a vector, wherein said vector includes a nucleic acid molecule which encodes for fibrinogen E for use in the recombinant manufacture of fibrinogen E.

Alternatively, vector(s) which include nucleic acid encoding polypeptides which comprise fibrinogen E can be engineered for recombinant expression.

In a preferred embodiment of the invention said vector is an expression vector adapted for prokaryotic or eukaryotic cell expression.

Typically said adaptation includes, by example and not by way of limitation, the provision of transcription control sequences (promoter sequences) which mediate cell/tissue specific expression. These promoter sequences may be cell/tissue specific, inducible or constitutive.

Promoter is an art recognised term and, for the sake of clarity, includes the following features which are provided by example only, and not by way of limitation. Enhancer elements are cis acting nucleic acid sequences often found 5′ to the transcription initiation site of a gene (enhancers can also be found 3′ to a gene sequence or even located in intronic sequences and is therefore position independent). Enhancers function to increase the rate of transcription of the gene to which the enhancer is linked. Enhancer activity is responsive to trans acting transcription factors (polypeptides) which have been shown to bind specifically to enhancer elements. The binding/activity of transcription factors (please see Eukaryotic Transcription Factors, by David S Latchman, Academic Press Ltd, San Diego) is responsive to a number of environmental cues which include, by example and not by way of limitation, intermediary metabolites (eg glucose, lipids), environmental effectors (eg light, heat,).

Promoter elements also include so called TATA box and RNA polymerase initiation selection (RIS) sequences which function to select a site of transcription initiation. These sequences also bind polypeptides which function, inter alia, to facilitate transcription initiation selection by RNA polymerase.

Adaptations also include the provision of selectable markers and autonomous replication sequences which both facilitate the maintenance of said vector in either. the eukaryotic, cell or prokaryotic host. Vectors which are maintained autonomously are referred to as episomal vectors. Episomal vectors are desirable since these molecules can incorporate large DNA fragments (30-50 kb DNA). Episomal vectors of this type are described in WO98/07876.

Adaptations which facilitate the expression of vector encoded genes include the provision of transcription termination/polyadenylation sequences. This also includes the provision of internal ribosome entry sites (IRES) which function to maximise expression of vector encoded genes arranged in bicistronic or multi-cistronic expression cassettes.

These adaptations are well known in the art. There is a significant amount of published literature with respect to expression vector construction and recombinant DNA techniques in general. Please see, Sambrook et al (1989) Molecular Cloning: A Laboratory Manual, Cold Spring Harbour Laboratory, Cold Spring Harbour, N.Y. and references therein; Marston, F (1987) DNA Cloning Techniques: A Practical Approach Vol III IRL Press, Oxford UK; DNA Cloning: F M Ausubel et al, Current Protocols in Molecular Biology, John Wiley & Sons, Inc.(1994).

Alternatively, or preferably, fibrinogen for use in the manufacture of fibrinogen E is isolated from natural sources using standard protein purification techniques well known in the art. Additionally, fibrinogen can be isolated from animal sources, other than human, for example pig.

According to a further aspect of the invention there is provided a method for the production of fibrinogen E comprising:
i) purifying fibrinogen from an animal;
ii) incubating said fibrinogen polypeptide with an effective amount of a protease capable of cleaving fibrinogen to provide at least fibrinogen E; and
iii) purifying fibrinogen E from fibrinogen and/or fibrinogen proteolytic fragments.

In a preferred method of the invention said fibrinogen is of human origin.

In a further preferred method of the invention said protease is plasmin.

According to a further aspect of the invention there is provided a method for the recombinant production of polypeptides comprising fibrinogen E including:
i) providing a cell transformed/transfected with vector(s) including fibrinogen E nucleic acid;
ii) providing conditions conducive to the manufacture of recombinant fibrinogen E polypeptides; and,
iii) purifying said fibrinogen E polypeptides from a cell, or a cells culture environment.

In a preferred method of the invention fibrinogen E polypeptides are provided with a signal sequence which facilitates the secretion of said polypeptides from said cell.

According to yet a further aspect of the invention there is provided a method to assemble fibrinogen E comprising:
i) providing quantities of polypeptides which from fibrinogen E;
ii) providing conditions conducive to the assembly of at least fibrinogen E; and
iii) purifying at least assembled fibrinogen E from the unassembled polypeptides.

According to yet still a further aspect of the invention there is provided a cell line transformed/transfected with at least one vector according to the invention wherein said vector includes nucleic acid molecules which encode polypeptides which comprise fibrinogen and/or fibrinogen E.

It will be apparent to one skilled in the art that the recombinant production of fibrinogen E will be facilitated by providing cell expression systems adapted to produce and assemble fibrinogen and/or fibrinogen E.

According to yet still a further aspect of the invention there is provided a non-human, transgenic animal characterised in that said animal incorporates at least one fibrinogen gene into its genome wherein the expression of said fibrinogen transgene is facilitated.

It will be apparent to one skilled in the art that the provision of non-human transgenic animals genetically modified by the provision of a fibrinogen transgene(s) is an alternative source of fibrinogen. It is well known in the art that transgenic animals can be used to make various therapeutic polypeptides.

In a preferred embodiment of the invention said fibrinogen transgene is of human origin.

In a further aspect of the invention there is provided a method to treat an animal which would benefit from inhibition of angiogenesis comprising:
i) administering an effective amount of an agent comprising fibrinogen E to an animal to be treated;
ii). monitoring the effects of said fibrinogen E on the inhibition of angiogenesis.

In a preferred method of the invention said treatment is the inhibition of tumour development.

In an alternative method of treatment polypeptides according to the invention are additionally conjugated, associated or crosslinked to an agent which augments the anti-angiogenic effect.

For example, a gene therapy vector includes a nucleic acid encoding a polypeptide according to the invention and a further nucleic acid encoding an anti-angiogenic agent.

Typically the agent could be a cytotoxic agent, another anti-angiogenic agent, a prodrug activating enzyme, a chemotherapeutic agent, a pro-coagulant agent or immunomodulatory factor. Examples of these are well known in the art, for example, and not by way of limitation cytotoxins, such as ricin A-chain or diphtheria toxin; antagonists of the key pro-angiogenic factors in tumours (eg VEGF, bFGF, TNF alpha, PDGF) would include neutralising antibodies or receptors for these factors, or tyrosine kinase inhibitors for their receptors (eg. SU5416 for the VEGF receptor, Flk1/KDR); prodrug activating enzymes such as, human simplex virus-thymidine kinase HSV-TK, which activates the prodrug, ganciclovir when it is then administered sytemically; chemotherapeutic agents, such as neocarzinostatin.

In addition, or alternatively, the cell surface domain of human tissue factor (this truncated form of tissue factor (tTF) could also be associated with polypeptides according to the invention. Truncated TF has limited anti-endothelial activity when free in the circulation, but becomes an effective and selective thrombogen (ie it causes extensive thrombosis and coagulation in blood vessels) when targeted to the surface of tumor endothelial cells,

An example of an immunomodulatory factor is the Fc effector domain of human IgG1. This binds natural killer (NK) cells and also the C1q protein that initiates the complement cascade. NK cells and complement then activate a powerful cytolytic response against the targeted endothelial cells.

It will be apparent that the above combinations of polypeptides and therapeutic agents will also have benefit with respect to the treatment of other conditions/diseases which are dependent on angiogenesis as herein disclosed.

According to a yet further aspect of the invention there is provided an imaging agent comprising a polypeptide according to the invention.

It will be apparent to the skilled artisan that polypeptides according to the invention can be used to target imaging agents to, for example, tumours, to identify developing tumours or to monitor the effects of treatments to inhibit tumour growth. It will also be apparent that the combined therapeutic compositions which comprise both polypeptides according to the invention and a further anti-angiogenic agent may be further associated with an imaging agent to monitor the distribution of the combined therapeutic composition and/or to monitor the efficacy of said combined composition.

Methods used to detect imaging agents are well known in the art and include, by example and not by way of limitation, positron emission tomographic detection of F¹⁸and C¹¹ compounds.

An embodiment of the invention will now be described, by example only, and with reference to the following figures:

FIG. 1 represents the amino acid sequences of the α-β-γ-polypeptides of fibrinogen E;

FIG. 2 represents a schematic illustration of the role of the enzymes, plasmin and thrombin, in the generation of the fibrin(ogen) breakdown products. Fibrinogen consists of three pairs of polypeptide chains α, β and γ joined by disulphide bonds to form a symmetric dimeric structure. The NH₂-terminal regions of all six chains form the central E-domain. This fibrinogen molecule, when cleaved by plasmin, releases two D-fragments (the COOH-terminal regions), one E-fragment and several smaller fragments including a small peptide, beta 1-42 (the amino terminal of the β chain). Cleavage by thrombin releases the two fibrinopeptides A and B (Fp A and B) from the NH₂-termini of α- and β-chains respectively, exposing polymerisation sites, which form electrostatic bonds between the E-domain of one molecule and the D-domain of an adjacent one resulting in lateral polymerisation of fibrin monomers into a fibrin polymer. Factor XIIIa, a transglutaminase, then introduces γ-glutamyl-ε-amino-lysine isopeptide cross-links between D-domains of. adjacent fibrin polymers stabilising the polymer into crosslinked fibrin which is more resistant to cleavage. This can then be broken down by plasmin cleavage in the three stranded coils found between the D and E-domains yielding D-dimer, D-fragment and FnE-fragment (which lacks the fibrinopeptides A and B) and smaller fragments (4);

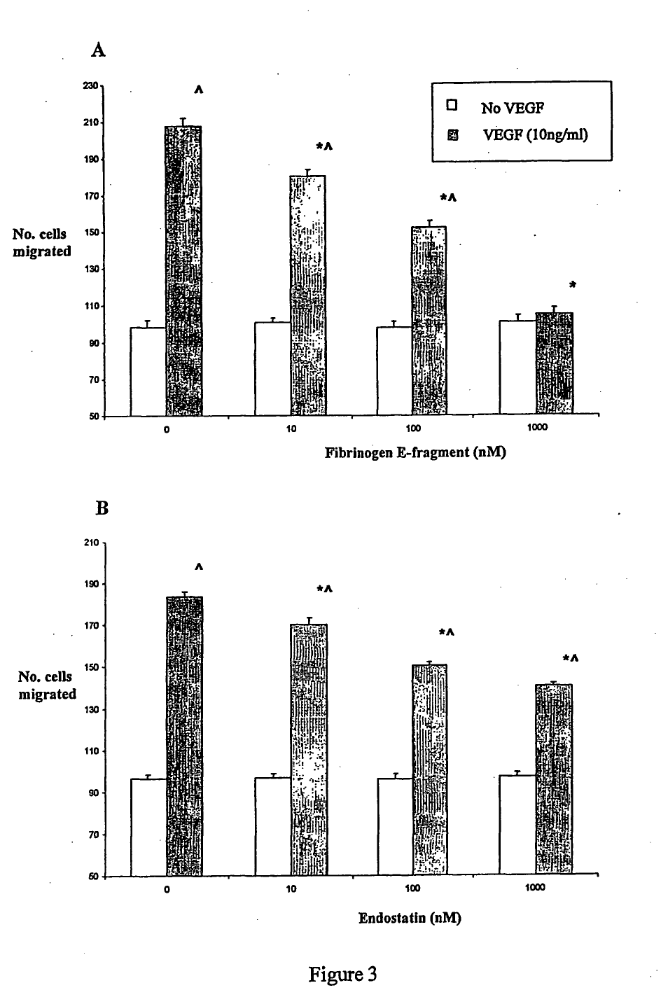

FIG. 3 represents mean (±SEM) number of human dermal microvascular endothelial cells (HuDMEC) migrating across a collagen-coated filter in response to control medium (no VEGF) or medium containing 10 ng/ml VEGF in the absence or presence of various concentrations of FgE-fragment (A) or endostatin (B). Representative data from 1 experiment are given as similar results were obtained in a further two identical experiments, and when VEGF was replaced by 10 ng/ml bFGF (data not shown). *P<0.001 compared to positive control (VEGF alone); ˆP<0.01 compared to negative control (no VEGF);

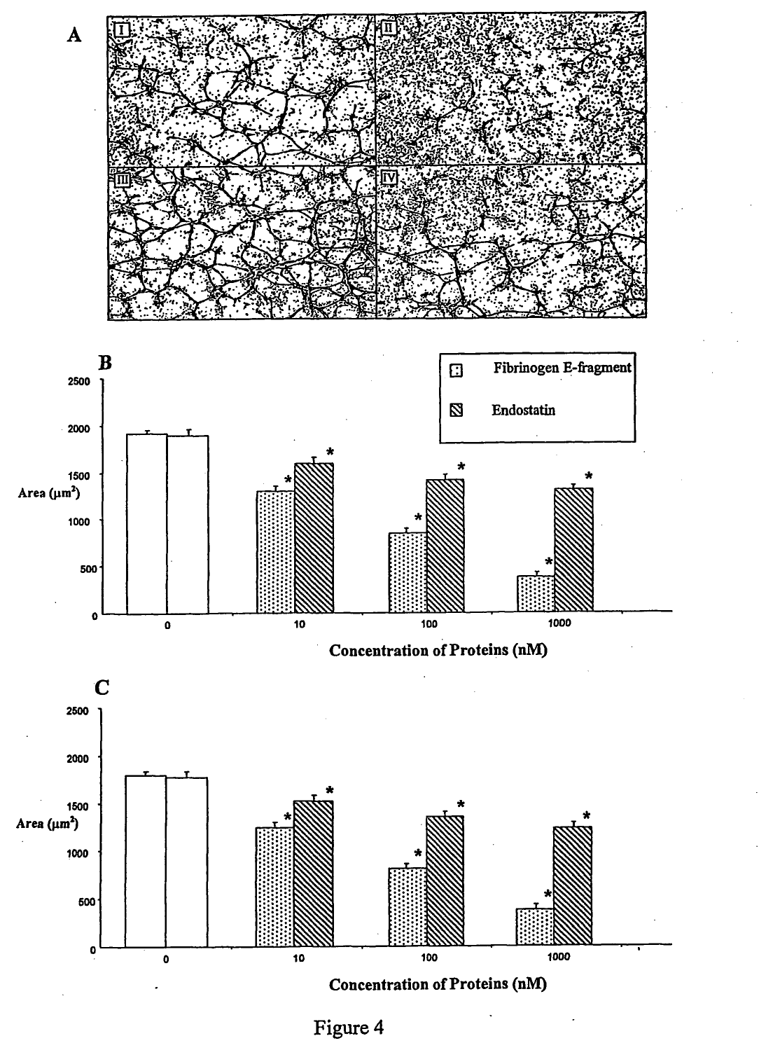

FIG. 4 represents, upper panel (A): Tubule formation in the growth factor (GF)-reduced Matrigel assay (×40 objective) in the absence of exogenous factors (I), or the presence of 100 nM FgE-fragment (II), 10 ng/ml. VEGF (III), or 100 nM endostatin (IV). Lower panels: mean (±SEM) area of tubule formation in the absence (empty bars) or presence of various concentrations of FgE-fragment or endostatin (shaded bars). HuDMECs were grown on GF-reduced Matrigel in DMEM+1%FCS with either VEGF (10 ng/ml) (Panel B) or bFGF (10 ng/ml) (Panel C). Representative data from 1 experiment are given as essentially similar results were obtained in 3 identical experiments. *P<0.04 compared to control group. P≦0.02 compared to same dose of fibrinogen E;

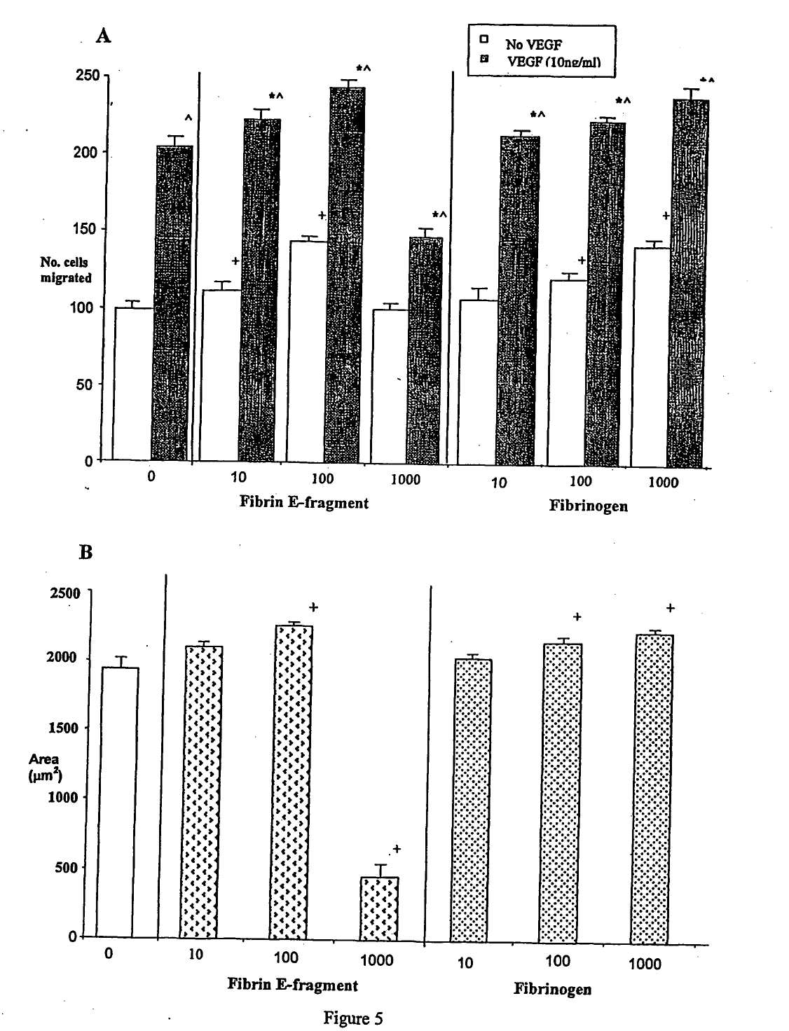

FIG. 5 effects of various fibrinogen breakdown products; fibrin E-fragment (FnE), and whole fibrinogen, on HuDMEC migration (panel A) or tubule formation in the GF-reduced Matrigel assay assessed as area (panel B) in the absence or presence of 10 ng/ml VEGF. Data are provided as means (±SEM) and all doses cited are in nM. Representative data from 1 experiment are given as similar results were obtained in a further two identical experiments (and a further set of 3 experiments in which VEGF was replaced by 10 ng/ml bFGF). *P<0.0, 01 compared to respective group (ie. either with or without fibrinogen or FnE-fragment) with no VEGF, ˆP<0.1 compared to respective group (ie. either with our without VEGF) with no fibrinogen or FnE-fragment;

FIG. 6 represents the DNA and protein sequence of the α-β-γ-chains of the fibrinogen E-fragment; and

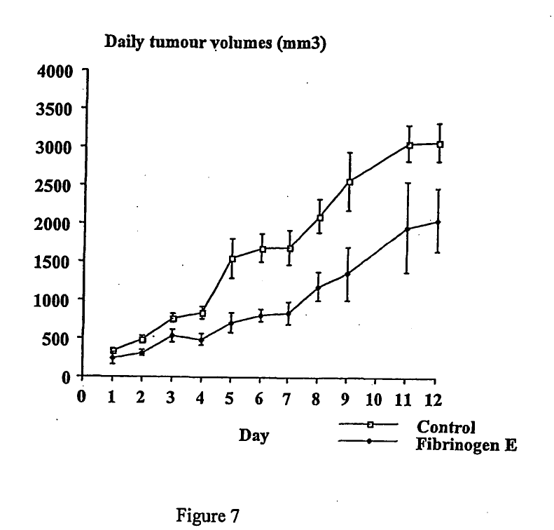

FIG. 7 illustrates the in vivo efficacy of fibrinogen E-fragment on tumour growth in mice.

### MATERIALS AND METHODS

Cell culture. Adult human dermal microvascular endothelial cells (HuDMECs) were obtained commercially (TCS Biologicals, Buckinghamshire, United Kingdom) and cultured in microvascular endothelial cell growth medium (EGM) containing heparin (10 ng/ml), hydrocortisone, human epidermal growth factor (10 ng/ml), human fibroblast growth factor (10 ng/ml) (such endothelial growth factors are necessary for routine passaging of HuDMECs in culture) and dibutyryl cyclic AMP. This was supplememented with 5% heat-inactivated FCS, 50 μg/ml gentamicin and 50 ng/ml amphotericin B (TCS Biologicals, United Kingdom). Cells were grown at 37° C. in a 100% humidified incubator with a gas phase of 5% CO₂ and routinely screened for Mycoplasma. Prior to their use in the assays indicated below, HuDMECs were grown to 80% confluency, incubated in DMEM+1%FCS for 2 h, then harvested with 0.05% trypsin solution, washed twice and resuspended at the required cell density.

### Proteins and Peptides

Commercial human fibrinogen (plasminogen/plasmin and thrombin free) was obtained from Enzyme Research Laboratories (Swansea, United Kingdom). The fibrinogen did not clot at any point during the experiments indicating that there was no activity within the preparation to change its conformation. Human fibrinogen E-fragment was purchased from Diagnostica Stago, Asnieres, France (produced by plasmin cleavage of fibrinogen and purified by electrophoresis, immunoelectrophoresis, ion exchange and gel filtration). To generate human fibrin E-fragment, fibrinogen E-fragment was digested with human thrombin (Sigma-Aldrich Co, Dorset, United Kingdom), as previously described. To control for the possible effects of trace amounts of thrombin in the Fn E fragment preparation on our assays, the same amount of thrombin (0.5 U/ml) was added to control media used in experiments using Fn E-fragment. HPLC-purified FpA was obtained commercially from Bachem. Ltd, Saffron Walden, UK. This peptide was included in the study as the amino termini of the two α fragments are retained in the Fgn E-fragment, but missing in the fibrin E-fragment. (ie as the FpA portion of this is missing). We, therefore, compared the effects of equimolar amounts of FpA and Fgn E fragment in the assays described below to ascertain whether effects induced by Fgn E-fragment were due to an active site located in the FpA part of the molecule. Human recombinant endostatin was obtained from Calbiochem, La Jolla, Calif.

### Migration Assay

The Boyden chamber technique was adapted from (13) and used to evaluate HuDMEC migration across a porous membrane towards a concentration gradient of either VEGF (10 ng/ml) or bFGF (10 ng/ml). The Neuro Probe 48 well microchemotaxis chamber (Neuro Probe Inc, Cabin John, Md.) was used with 8 μm pore size polycarbonate membranes (Neuro Probe Inc, Cabin John, Md.) coated with 100 μg/ml collagen type IV. 10 ng/ml VEGF or bFGF alone or with various concentrations of fibrinogen, fibrinogen E-fragment, fibrin E-fragment, or fibrinopeptide A were dissolved in DMEM+1%FCS and placed in the lower wells. The collagen-coated membrane was then placed over this and 50 μl of 25×10⁴ HuDMECs/ml (in DMEM containing 1%FCS) added to the upper chamber. The chambers were then incubated at 37° C. for 4.5 h. The chamber was then dismantled, the membrane removed and non-migrated cells scraped off the upper surface. Migrated cells on the lower surface were fixed with methanol, stained with Hema ‘Gurr’ rapid staining kit (Merck, Leics, United Kingdom) and counted using a light microscope (×160 magnification) in 3 random fields per well. Each test condition was carried out in 3-6 replicate wells and each experiment repeated 3 times.

### Tubule Formation Assay

24 well plates were coated with 30 μl/well of growth factor-reduced (GF-reduced) Matrigel (Becton Dickinson Labware, Bedford, Mass.). Endothelial cells plated on this matrix migrate and differentiate into tubules within 6 h of plating as described previously (14). HuDMECs were seeded at a density of 4×10⁴ cells/ml and incubated for 6 h in 500 μl of either DMEM+1%FCS alone (control), or this medium ±10 ng/ml VEGF or bFGF in the presence or absence of whole fibrinogen or one of the fibrin(ogen) degradation products. Assessment of tubule formation involved fixing the cell preparation in 70% ethanol at 4° C. for 15 minutes, rinsing in PBS and staining with. haematoxylin and eosin. Three random fields of view in 3 replicate wells for each test condition were visualised under low power (×40 magnification), and colour images captured using a Fuji digital camera linked to a Pentium III computer (containing a frame grabber board). Tubule formation was assessed by counting the number of tubule branches and the total area covered by tubules in each field of view using image analysis software supplied by Scion Image.

### Proliferation Assay

The MTT (3-[4,5-Dimethylthiazol-2-yl]-2,5-diphenyltetrazolium bromide) assay was used as previously described (12) to assess HuDMEC proliferation induced by VEGF or bFGF in the absence or presence of fibrinogen or a fibrin(ogen) breakdown product. HuDMEC were seeded at 3×10³ cells/100 μl in DMEM+1%FCS±10 ng/ml VEGF or bFGF in test solution into 96 well, microtitre plate for 4.5 and 6 h. At these time points, a quarter volume of MTT solution (2 mg MTT/ml PBS) was added to each well and each plate was incubated for 4 h at 37° C. resulting in an insoluble purple formazan product. The medium was aspirated and the precipitates dissolved in 100 μl DMSO buffered at pH 10.5. The absorbance was then read at 540 mm on a Dynex ELISA plate reader.

### Cytotoxicity Assay

HuDMECs were seeded at a density of 1-2×10⁵ cells per well in a 24 well-plate in the absence or presence of fibrinogen or a fibrin(ogen) degradation product. After 6 h, both live (following removal by trypsinisation) and dead (floating) cells were harvested and cell viability of all cells present assessed using propidium iodide staining of 5000 cells in each of triplicate samples per treatment using a FACScan (Becton Dickinson) equipped with a blue laser excitation of 15 mW at 488 mn. The data was collected and analysed using Cell Quest software (Becton Dickinson).

### In Vivo Efflicacy of Fibrinogen E Fragment

### Animals

Experiments were performed on six-week-old Balb/C mice weighing 15 g, obtained from Sheffield Field Laboratories. All experiments were approved by the Home Office Project Licence Number PPL50/1414.

### Tumour Cell Culture

The CT26 cell line was maintained by in vitro passage in Dulbecco's Minimal Eagles Medium containing 10% foetal calf serum, and 1% penicillin and streptomycin and maintained at 37° C. in humidified. atmosphere of 5% CO₂ in air. The cell line was routinely checked to ensure freedom from mycoplasma (Mycoplasma rapid detection system, Gena-Probe Incorporated, U.S.A.)

### Subcutaneous Tumour Implantation

Animals were anaesthetised with an intraperitoneal injection of diazeparn (0.5 mg/ml, Dumex Ltd.) and hypnorm (fentanyl citrate 0.0315 mg/ml and fluanisone 1 mg/ml, Janssen Pharmaceutical Ltd.) in the ratio of 1:1 at a volume of 0.1 ml/200 g body weight, with supplementation as required to maintain adequate anaesthesia. Naïve Balb/c mice were immunised s.c into the right flank, following removal of the fur. Tumour cells were injected at a concentration of 3×10⁵ viable CT26 cells per animal suspended in 100 ul serum free medium. Animals were then allowed to recover. Tumour growth and animal weights were monitored daily.

### Administration of Fibrinogen E Fragment

Tumour growth was measured daily and when the majority of animals in the cohort had tumour volumes of >100 mm³ but <350 mm³ animals were divided into experimental and control groups. This occurred between 14 and 18 days following implantation of the tumour cell suspension. Animals then received an intraperitoneal (ip) injection of either active drug (fibrinogen E fragment 100 Mm) or vehicle (phophate buffered saline, μl). Daily injections continued until the tumour growth in the control animals reached the maximum burden allowed by Home Office legislation.

### Assessment of Tumour Growth

Tumour volumes were assessed by claiper measurments of the perpendicular diameters and vulumes estimated using the equation:
Volume=(a²×b)/2
where a is the smaller and b the larger diameter.

Animals were weighed on a daily basis and the general well being monitored.

### Statistical Analysis

All experiments were performed at least three times and data analysed using the Mann-Whitney U test, a non-parametric test that does not assume a Gaussian distribution in the data being analysed. P≦0.05 was taken as significant.

### Results and Discussion

HuDMECs were seen to migrate across collagen-coated filters in the chemotaxis assay and form tubules on GF-reduced Matrigel in the absence of exogenous stimuli (although it should be noted that a residual level of growth factors is present even in GF-reduced Matrigel). Both cell activities were significantly (P<0.001) increased in the presence of 10 ng/ml VEGF (FIGS. 3: A&B, 4A: photographs I & III, 5: A). Exposure to fibrinogen E-fragment (FgE-fragment) significantly (P<0.001) inhibited both VEGF-induced migration (FIG. 3A) and tubule formation, as assessed by either total tubule area or the number of branches of HuDMECs in a dose-dependent manner (FIGS. 4A: photograph II and 4B). None of the doses of FgE-fragment tested in this study altered cell migration in the absence of VEGF (FIG. 3A). The inhibitory effects of FgE-fragment were not due to a cytostatic or cytotoxic effect of this molecule at 10 and 100 nM, as neither concentration had any notable effect on HuDMEC proliferation or viability (in control medium or medium containing 10 ng/ml VEGF; data not shown) in our assay systems. However, the marked decrease in HuDMEC migration and tubule formation evident at 1 μM FgE-fragment may have been due, at least in part, to a cytotoxic effect, as this dose resulted in a marginal but significant (P<0.05) reduction in the viability and proliferation of HuDMECs (data not shown).

Essentially similar results were obtained in these studies when VEGF was replaced by 10 ng/ml bFGF (FIG. 4C), indicating that FgE-fragment inhibits HuDMEC activity at a post-VEGF receptor locus common to both VEGF and bFGF signalling in endothelial cells. The putative receptor(s) that bind FgE-fragment on endothelial cells have yet to be defined, although Dejana et al (13) indicated that FgE-fragment may be capable of binding the fibrinogen receptor in vitro. However, RGD motifs in the D-domains of the fibrinogen molecule mediate binding of this protein to the fibrinogen receptor (14). These sites are absent in FgE-fragment so binding to the fibrinogen receptor would involve a novel, non-RGD region of this fragment. It is not known whether this receptor is involved in the inhibitory effects of FgE-fragment demonstrated here and a distinct receptor/signalling pathway may be involved. It is to be noted that the 130 kD endothelial cell receptor binding site B β 15-42 (Erban and Wagner J Biol Chem 267, 2451-2458, 1992; Bach et al J Biol Chem 273, 30719, 1998) is absent from the fibrinogen E-domain and therefore not directly involved in the anti-angiogenic effect.

It could be argued that the inhibitory effects of FgE-fragment are due to an indirect rather than a direct effect on endothelial cells, as there is no effect seen on non-stimulated endothelial cells. For example, fibrinogen has recently been shown to be capable of binding to such pro-angiogenic factors as bFGF (15), and could thereby block the pro-angiogenic function(s) of such cytokines. It is not known, however, whether fibrinogen can also bind VEGF or whether FgE-fragment, like its parent molecule, can bind either growth factor. It was possible that FgE-fragment may bind non-specifically to the filter in the chemotaxis assay and/or constituents of the Matrigel matrix in the tubule formation assay, thereby reducing endothelial cell adhesion and function. As one or both of these could, in theory, be responsible, wholly or in part, for the inhibition of HuDMEC migration and tubule formation by FgE-fragment recorded in this study, we repeated these studies and pre-exposed endothelial cells to FgE-fragment prior to their use in these assays. Exposure of HuDMECs to 10 and 100 nM FgE for 1 h was sufficient to cause virtually the same level of inhibition in VEGF/bFGF-induced migration and tubule formation as that seen when FgE-fragment was present throughout the assay (data not shown).

In order to assess the anti-angiogenic potential of FgE-fragment, the level of endothelial cell inhibition was compared with that elicited by the well-characterised anti-angiogenic agent, endostatin. Others have reported that 700 ng/ml (35 nM) endostatin is highly effective in blocking angiogenesis in vitro (16), so various concentrations in this range were used in the present study. FgE-fragment produced similar or greater levels of inhibition than seen by any concentration of endostatin (FIGS. 3B and 4A: photograph IV and 4B&C). This finding suggests that, whatever the mechanism subserving its effect, FgE-fragment is a potent, new antagonist of angiogenic growth factors in vitro.

It may be important to note that the effects of FgE-fragment are not confined to endothelial cells. This polypeptide is known to also inhibit the migratory activity of neutrophils (17), stimulate fibrinogen release by hepatoctyes (18), and enhance the release of IL-6 by macrophages (19). Further studies are required to see whether these and possibly other effects of FgE-fragment, as yet undefined, will result in limiting side effects during or after its administration in vivo.

The anti-angiogenic effects of FgE-fragment contrast with results obtained using equimolar amounts of fibrinogen, fibrin E-fragment (FnE) and fibrinopeptide A. Both fibrinogen and fibrin E-fragment (FnE) significantly (P<0.001) increased control and VEGF-induced migration of HuDMECs at doses of 100 nM (FIG. 5A). Furthermore, both 100 nM fibrin E-fragment and 100 nM and 1 μM fibrinogen significantly (P<0.05) enhanced tubule formation (FIG. 5B). This accords well with previous reports showing that fibrinogen stimulates endothelial cell migration (13). Fibrin E-fragment has also been shown to be pro-angiogenic, possibly due to conformational changes induced within the fragment by thrombin cleavage of fibrinopeptide A. 10 nM and 100 nM fibrin E-fragment also appeared to increase the proliferation rate of HuDMECs. However, as with fibrinogen E-fragment, the highest dose (1 μM) of fibrin E-fragment was cytotoxic for HuDMECs and triggered a significant (P<0.001) decrease in cell viability and proliferation (data not shown). This in turn caused marked reductions in HuDMEC migration and tubule formation in our assays systems (FIG. 5A and B). Similar results were obtained when VEGF was replaced by 10 ng/ml bFGF in these assays.

Fibrinogen E- and fibrin E-fragments differ in that the latter is denuded of fibrinopeptide A by thrombin cleavage. We, therefore, investigated whether the anti-angiogenic function of fibrinogen E-fragment resides in this part of the molecule by testing the effects of equimolar amounts of fibrinopeptide A alone on HuDMEC migration and tubule formation. This was not seen to exert a significant effect on such endothelial cell activities, suggesting that the active moiety resides in the central E domain of the molecule, or requires part or all of the rest of the domain, or resides in the FpA part of the amino terminus of the a chain but is only held in the correct conformation for biological activity when it is attached to the rest of the fragment.

We have also investigated the in vivo effect of the fibrinogen E fragment.

### EXAMPLE 1

This initial pilot study investigated the effects of fibrinogen E fragment (100 mM) or vehicle administered daily i.p. for 10 days in two groups of mice (n=3). Starting tumour volume was less than 100 mm³ in both groups. Tumours in the control group continued to grow at a steady rate over the ten day study period and reached a final tumour volume of 590±120 mm³ when the animals were killed at 10 days after commencing the injections. In contrast, tumours in the experimental group continued to grow at a similar rate to the control tumours until Day 5 (300 mm³) when the growth rate stabilised for the remaining period of the study.

### EXAMPLE 2

The initial pilot study was repeated with an experimental group (n=8) receiving 100 mM Fibrinogen E fragment ip and control group (n=7) receiving vehicle daily for 12 days. Starting tumour volume was less than 350 mm³ in both groups. Tumours in the control group continued to grow steadily over the 12 day period reaching a final tumour volume of 3072±255 mm . In contrast tumours in the experimental animals had a reduced but steady rate of growth with a final tumour volume of 2052±414 mm³ (p<0.001).

This data, see FIG. 7, therefore demonstrates the potential of Fibrinogen E fragment as an in vivo anti-angiogenic agent.

### REFERENCES

1. Folkman J Angiogenesis in cancer, vascular, rheumatoid and other disease. Nature Medicine, 1: 27-31, 1995.
2. Leek R, Harris A L, and Lewis C E Cytokine networks in solid human tumours: regulation of angiogenesis. J Leuk. Biol., 56: 423-35, 1994.
3. Cao Y Endogenous angiogenesis inhibitors: angiostatin, endostatin, and other proteolytic fragments. Prog Mol Subcell Biol., 20:161-76, 1998.
4. Doolittle R Fibrinogen and Fibrin. Scientific American, 245: 92-101, 1981.
5. Costantini V, Zacharski L R, Memoli-VA, Kisiel W, Kudryk B J, and Rousseau S M Fibrinogen deposition without thrombin generation in primary human breast cancer tissue. Cancer Res., 51:3 49-53, 1991.
6. Dvorak H F, Nagy J A, Feng D, Brown L F and Dvorak A M Vascular permeability factor/vascular endothelial growth factor and the significance of microvascular hyperpermeability in angiogenesis. Curr Top Microbiol Immunol, 237:97-132, 1999.
7. Thompson W D, Wnag J E H, Wilson S J, and Ganesalingbam N Angiogenesis and fibrin degradation in human breast cancer. Angiogenesis: Molecular Biology, Clinical Aspects, 245-251, 1994.
8. Thompson W D, Smith E B, Stirk C M, Marshall Fl, Stout A J, and Kocchar A Angiogenic activity of fibrin degradation products is located in fibrin fragment E. J. Pathol, 168: 47-53, 1992.
9. Malinda K M, Ponce L, Kleinman H K, Shackelton L M, and Millis A J Gp38k, a protein synthesized by vascular smooth muscle cells, stimulates directional migration of human umbilical vein endothelial cells. Exp Cell Res 250:168-73, 1999.
10. Shen J, Ham R G, Karmiol S Expression of adhesion molecules in cultured human pulmonary microvascular endothelial cells. Microvasc Res., 50:360-72, 1995.
11. Liu J, Kolath J, Anderson J, Kolar C, Lawson T A, Talmadge J, and Gmeiner W H Positive interaction between 5-FU and FdUMP[10] in the inhibition of human colorectal tumour cell proliferation. Antisense Nucleic Acid Drug Dev., 9(5):481-6, 1999.
12. Dejano E, Languino L R, Polentarutti N, Balconi G, Ryckewaort J J, Larrieu M J, Donati M B, Mantovani A, and Marguerie G Interaction between fibrinogen and cultured endothelial cells. J. Clin. Invest., 75: 11-18, 1985.
13. Suehiro K, Gailit J, Plow E F Fibrinogen is a ligand for integrin alpha5beta1 on endothelial cells. J. Biol. Chem., 272:5360.6, 1997. 20
14. Sahni A, Odrljin T, and Francis C W Binding of basic fibroblast growth factor to fibrinogen and fibrin. J Biol Chem., 273:7554-9, 1998.
15. Taddei L, Chiarugi P, Brogelli L, Cirri P, Magnelli L, Raugei G, Ziche M, Granger H J, Chiarugi V, and Ramponi G Inhibitory effect of full-length human endostatin on in vitro angiogenesis. Biochem Biophys Res Commun., 263:340-5, 25 1999.
16. Kazura J W, Wenger J D, Salata R A, Budzynski A Z, and Goldsmith G H Modulation of polymorphonuclear leukocyte microbicidal activity and oxidative metabolism by fibrinogen degradation products D and E. J Clin Invest., 83:1916-24, 1989.
17. Qureshi G D, Guzelian P S, Vennart R M, and Evans H J Stimulation of fibrinogen synthesis in cultured rat hepatocytes by fibrinogen fragment E. Biochim Biophys Acta, 844:288-95, 1985.
18. Lee M E, Rhee K J, and Nham S U Fragment E derived from both fibrin and fibrinogen stimulates interleukin-6 production in rat peritoneal macrophages. Mol Cells, 9:7-13, 1999.

1-10. (canceled)

11. The use of a fibrinogen E polypeptide for the manufacture of a medicament for use in the treatment of cancer.

12-22. (canceled)

23. A method to treat a human or an animal which would benefit from inhibition of angiogenesis comprising:
i) administering an effective amount of a fibrinogen E polypeptide to the animal; and optionally
ii) monitoring the effects of the polypeptide on the inhibition of angiogenesis.

24. A method for inhibiting tumor development comprising:
i) administering an effective amount of a fibrinogen E polypeptide to a human or animal; and optionally
ii) monitoring the effects of the polypeptide on the inhibition of tumour development.

25-26. (canceled)
